(19) United States
(12) Patent Application Publication
(10) Pub. No.: US 2007/0155752 A1
(43) Pub. Date: Jul. 5, 2007

(54) 2-Amino-o4-substituted pteridines and their use as inactivators of o6-alkylguanine-dna alkyltransferase

(75) Inventors: Rorbert C. Moschel, Frederick, MD (US); Michael E. Nelson, Derwood, MD (US); Anthony E. Pegg, Hershey, PA (US); Natalia A. Loktionova, Elizabeth Town, PA (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignees: Government of the United States of America,; Dept of Health and Human Services, Rockville, MD (US); THe Penn State Research Foundation, University Park, PA (US)

(21) Appl. No.: 10/585,566

(22) PCT Filed: Dec. 10, 2004
(86) PCT No.: PCT/US04/41577

§ 371 Date: Aug. 29, 2006

Related U.S. Application Data
(60) Provisional application No. 60/534,519, filed on Jan. 6, 2004.

(51) Int. Cl. A61K 31/525; C07D 475/02

(52) U.S. Cl. 514/251; 544/258

(57) ABSTRACT

Disclosed are pteridine derivatives of formula (I): (I), wherein, for example, R₁ and R₂ are hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxyalkyl wherein the alkyl is C₁-C₆, halogen, or hydroxy, or a group of formula II: (II); and R₃ is (a) phenyl or (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, the cyclic group optionally with a carbocyclic or heterocyclic ring fused thereto, which is substituted with 1 to 5 substituents. Disclosed also are pharmaceutical compositions, a method of enhancing the chemotherapeutic effectiveness of cancer treatment agents, a method of deactivating the O⁶-alkylguanine-DNA alkyltransferase enzyme, and a method of inhibiting the reaction of O⁶-alkylguanine-DNA alkyltransferase enzyme with an alkylated DNA.

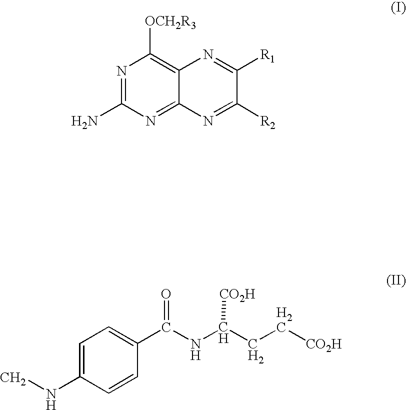

### FIELD OF THE INVENTION

This invention pertains to certain pteridine derivatives, pharmaceutical compositions comprising such derivatives, and their use as inactivators of the O⁶-alkylguanine-DNA alkyltransferase protein (“AGT” or “alkyltransferase”). These derivatives are contemplated for use in conjunction with cancer treatment agents such as carmustine, lomustine, nimustine, or temozolomide for enhancing the chemotherapeutic efficacy of these cancer treatment agents.

### BACKGROUND OF THE INVENTION

O⁶-Belizylguanine derivatives, ^{1,2} some O⁶-benzylpyrimidines³ and related compounds^{4,5} are known to be inactivators of the human DNA repair protein, AGT.⁶ See also U.S. Pat. Nos. 5,091,430; 5,352,669; 5,358,952; 5,525,606; 5,691,307; 5,753,668; 5,916,894; 5,958,932; 6,172,070; 6,303,604; 6,333,331; and 6,436,945. This repair protein is the primary source of resistance many tumor cells exhibit to chemotherapeutic agents that modify the O⁶-position of DNA guanine residues.⁶ Therefore, inactivation of this protein can bring about a significant improvement in the therapeutic effectiveness of these chemotherapy drugs. The prototype inactivator, O⁶-benzylguanine is currently in clinical trials in the US as an adjuvant in combination with the chloroethylating agent, 1,3-bis(2-chloroethyl)-1-nitrosourea (BCNU) and the methylating agent, temozolomide.^{7,8} O⁶-(4-bromothenyl)guanine is in clinical trials in the UK⁹ as an alkyltransferase inactivator. There is a desire, and therefore, a need, in the pharmaceutical industry for novel inactivators that have one or more advantageous properties compared to O⁶-benzylguanine or O⁶-(4-bromothenyl)guanine such as improved water solubility and/or greater tumor selectivity. The advantages of this invention, as well as additional inventive features, will be apparent from the description of the invention provided herein.

### BRIEF SUMMARY OF THE INVENTION

The foregoing need has been fulfilled to a great extent by the present invention which provides pteridine derivatives of formula (I): 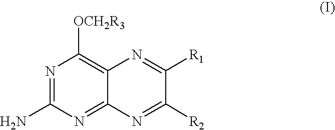
wherein R₁, R₂, and R₃ are suitable substituents. The present invention also provides pharmaceutical compositions comprising a pteridine derivative or pharmaceutically acceptable salt thereof. The present invention also provides a method of enhancing the chemotherapeutic effectiveness of cancer treatment agents by the use of these pteridine derivatives. The present invention further provides a method of deactivating or reducing the activity of AGT, as a well as a method of inhibiting the reaction of AGT with an alkylated DNA.

While the invention has been described and disclosed below in connection with certain embodiments and procedures, it is not intended to limit the invention to those embodiments. Rather, it is intended to cover all such alternative embodiments and modifications as fall within the spirit and scope of the invention

### BRIEF DESCRIPTION OF THE DRAWINGS

FIG. 1 depicts a reaction scheme useful to prepare certain compounds (4-7) in accordance with an embodiment of the invention. “Bn” in the formulas represents benzyl.

FIG. 2 depicts the effect of compound 7 on cell killing of various tumor cells by BCNU.

### DETAILED DESCRIPTION OF THE INVENTION

The present invention provides compounds having, in addition to AGT inactivating ability, one or more advantageous properties compared to O⁶-benzylguanine. Accordingly, the present invention provides compounds of formula (I): 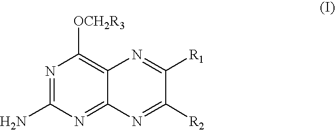
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl, and a group of formula (II): 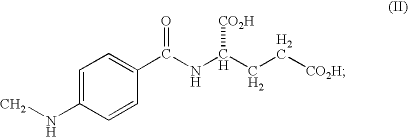
R₃ is (a) phenyl; (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, the cyclic group optionally with a carbocyclic or heterocyclic ring fused thereto, which is substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SO_{N}R′ wherein n=0, 1, 2 or 3, R′ is H, a C₁-C₆ alkyl or aryl; or a pharmaceutically acceptable salt thereof; with the provisos that (1) R₁ and R₂ are not simultaneously hydrogen; and (2) when R₃ is unsubstituted phenyl, R₁ and R₂ are not simultaneously methyl. “Halo” refers to fluoro, chloro, bromo, or iodo. “Aryl” refers to an aromatic group having 1, 2, or 3 phenyl rings.

In an embodiment of the invention, R₃ is phenyl or a phenyl group substituted with 1, 2, 3, 4, or 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently Cₗ-C₆, aminoalkyl wherein the alkyl is C₁-C₆, and SO_{N}R′ wherein n=0, 1, 2 or 3, R′ is H, a C₁-C₆ alkyl or aryl; or a pharmaceutically acceptable salt thereof.

In accordance with a preferred embodiment, the present invention provides compounds of formula (I), wherein R₁ is selected from the group consisting of hydrogen, C₁-C₆ alkyl, carboxyl, formyl, C₁-C₆ hydroxyalkyl, C₁-C₆ carboxyalkyl, C₁-C₆ formyl alkyl, and a group of formula (II) and R₂ is hydrogen or C₁-C₆ alkyl; and R₃ is phenyl or a substituted phenyl, wherein the substituents on phenyl are described above; or a pharmaceutically acceptable salt thereof.

In a more preferred embodiment, the present invention provides compounds of formula (I), wherein R₁ is selected from the group consisting of hydrogen, C₁-C₆ alkyl, C₁-C₆ hydroxyalkyl, carboxyl, formyl, and a group of formula (II), R₂ is hydrogen or C₁-C₆ alkyl, and R₃ is phenyl; or a pharmaceutically acceptable salt thereof. Particular embodiments of the compounds include those wherein R₁ is hydroxymethyl, carboxyl, formyl, or a group of formula (II), R₂ is hydrogen, and R₃ is phenyl; or a pharmaceutically acceptable salt thereof. Specific embodiments of the compounds of the present invention include compounds of formula (I), wherein R₁ is a group of formula (II), R₂ is hydrogen; and R₃ is phenyl; or a pharmaceutically acceptable salt thereof.

In certain embodiments, R₃ is a 5-membered ring containing N, S or O, with or without a second ring fused thereto; for example, R₃ is a heterocyclic ring having at least one S atom; e.g., a thiophene ring or a substituted derivative thereof. Alternatively, R₃ may be a heterocyclic ring having at least one O atom, particularly, a 5-membered heterocyclic ring having at least one O atom and more particularly R₃ may be a furan ring or a substituted derivative thereof. As another alternative, R₃ may be a heterocyclic ring having at least one N atom, particularly R₃ may be a 6-membered heterocyclic ring having at least one N atom and in particular, R₃ may be a pyridine ring. Examples of R₃ include halothiophenyl, i.e., chloro, bromo, fluoro, or iodo thiophene; the halo group can be at any suitable position, e.g., the 4-chloro or 4-bromo thiophene derivative. In embodiments, the carbocyclic or heterocyclic ring fused to the heterocyclic ring in R₃ may itself be bicyclic, e.g., naphthalene.

The present invention further provides pharmaceutical compositions comprising at least one of the compounds of the invention and a pharmaceutically acceptable carrier.

The present invention also provides a method of enhancing the chemotherapeutic treatment of tumor cells in a mammal with an antineoplastic alkylating agent that causes cytotoxic lesions at the O⁶-position of guanine, which method comprises administering to the mammal an effective amount of a compound of formula (I): 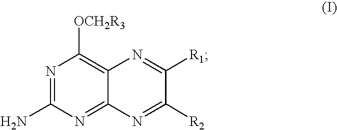
wherein R₁ and R₂ are independently selected from the group consisting hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl and a group of formula (II): 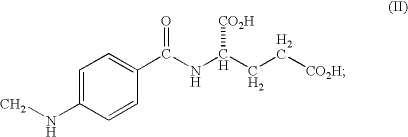
R₃ is (a) phenyl; (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, the cyclic group optionally with a carbocyclic or heterocyclic ring fused thereto, which is substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof, with the proviso that R₁ and R₂ are not simultaneously hydrogen; and administering to the mammal an effective amount of an antineoplastic alkylating agent which causes cytotoxic lesions at the O⁶-position of guanine

The present invention further provides a method for treating tumor cells in a mammal comprising administering to the mammal an amount effective to reduce the AGT activity in the mammal of a compound of formula (I): 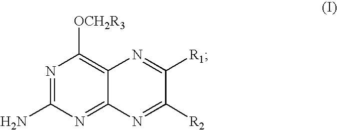
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, -nitro, C₃-C₈ cycloalkyl, C₂-C₆ alkenyl, C₂-C₆ alkynyl, and a group of formula (II): 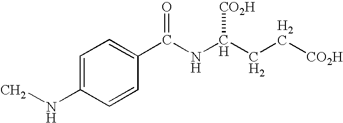
R₃ is (a) phenyl or (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, the cyclic group optionally with a carbocyclic or heterocyclic ring fused thereto, which is substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SO_{N}R′ wherein n=0, 1, 2 or 3, R′ is H, a C₁-C₆ alkyl or aryl; or a pharmaceutically acceptable salt thereof, with the proviso that R₁ and R₂ are not simultaneously hydrogen; and administering to the mammal an effective amount of an antineoplastic alkylating agent which causes cytotoxic lesions at the O⁶-position of guanine.

The compounds of the present invention can be administered in any suitable manner to a mammal for the purpose of enhancing the chemotherapeutic treatment of a particular cancer. Although more than one route can be used to administer a particular compound, a particular route can provide a more immediate and more effective reaction than another route. Accordingly, the described methods provided herein are merely exemplary and are in no way limiting.

Generally, the compounds of the present invention as described above will be administered in a pharmaceutical composition to an individual afflicted with a cancer. Those undergoing or about to undergo chemotherapy can be treated with the compounds separately, sequentially, simultaneously, or in conjunction with other treatments, as appropriate. In therapeutic applications, compositions are administered to a patient in an amount sufficient to elicit an effective depression of AGT activity thereby potentiating the cytotoxicity of the afore described chemotherapeutic treatment. An amount adequate to accomplish this is defined as a “therapeutically effective dose,” which is also an “AGT inactivating effective amount.” Amounts effective for a therapeutic or prophylactic use will depend on, e.g., the stage and severity of the disease being treated, the age, weight, and general state of health of the patient, and the judgment of the prescribing physician. The size of the dose will also be determined by the compound selected, method of administration, timing and frequency of administration as well as the existence, nature, and extent of any adverse side-effects that might accompany the administration of a particular compound and the desired physiological effect. It will be appreciated by one of skill in the art that various disease states may require prolonged treatment involving multiple administrations, perhaps using a series of different AGT inactivators and/or chemotherapeutic agents in each or various rounds of administration.

Suitable chemotherapeutic agents usefully administered in coordination with the compounds of the present invention include alkylating agents, such as chloroethylating and methylating agents. Such agents may be administered using conventional techniques such as those described in Wasserman et al., Cancer, 36, pp. 1258-1268 (1975), and Physicians' Desk Reference, 48th ed., Edward R. Barnhart publisher (1994). For example, 1,3-bis(2-chloroethyl)-1-nitrosourea (carmustine or BCNU, Bristol-Myers, Evansville, Ind.) may be administered intravenously at a dosage of from about 150 to 200 mg/m² every six weeks. Another alkylating agent, 1-(2-chloroethyl)-3-cyclohexyl-1-nitrosourea (lomustine or CCNU, Bristol-Myers), may be administered orally at a dosage of about 130 mg/m2 every six weeks. Other alkylating agents may be administered in appropriate dosages via appropriate routes of administration known to skilled medical practitioners.

Suitable doses and dosage regimens can be determined by conventional range-finding techniques known to those of ordinary skill in the art. Generally, treatment is initiated with smaller dosages that are less than the optimum dose of the compound. Thereafter, the dosage is increased by small increments until the optimum effect under the circumstances is reached. The present inventive method typically will involve the administration of about 1 mg to about 50 mg of one or more of the compounds described above per kg body weight of the individual. For a 70 kg patient, dosages of from about 10 mg to about 200 mg of the compound would be more commonly used, possibly followed by further lesser dosages from about 1 mg to about 50 mg of the compound over weeks to months, depending on a patient's physiological response, as determined by measuring cancer-specific antigens or other measurable parameters related to the tumor load of a patient.

It must be kept in mind that the compounds and compositions of the present invention generally are employed in serious disease states, that is, life-threatening or potentially life-threatening situations. In such cases, in view of the minimization of extraneous substances and the relative nontoxic nature of the compounds, it is possible and may be felt desirable by the treating physician to administer substantial excesses of these compounds.

Single or multiple administrations of the compounds can be carried out with dose levels and pattern being selected by the treating physician. In any event, the pharmaceutical formulations should provide a quantity of AGT-inactivating compounds of the invention sufficient to effectively enhance the cytotoxic impact of the chemotherapy.

The pharmaceutical compositions for therapeutic treatment are intended for parental, topical, oral or local administration and generally comprise a pharmaceutically acceptable carrier and an amount of the active ingredient sufficient to reduce, and preferably prevent, the activity of the AGT protein. The carrier may be any of those conventionally used and is limited only by chemico-physical considerations, such as solubility and lack of reactivity with the compound, and by the route of administration.

Examples of pharmaceutically-acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphanic, for example.

The pharmaceutically acceptable carriers described herein, for example, vehicles, adjuvants, excipients, or diluents, are well known to those who are skilled in the art and are readily available to the public. It is preferred that the pharmaceutically acceptable carrier be one that is chemically inert to the active compounds and one that has no detrimental side effects or toxicity under the conditions of use. Such pharmaceutically acceptable carriers preferably include water USP, saline (e.g., 0.9% saline), Cremophor EL (which is a derivative of castor oil and ethylene oxide available from Sigma Chemical Co., St. Louis, Mo.) (e.g., 5% Cremophor EL/5% ethanol/90% saline, 10% Cremophor EL/90% saline, or 50% Cremophor EL/50% ethanol), propylene glycol (e.g., 40% propylene glycol/10% ethanol/50% water), polyethylene glycol (e.g., 40% PEG 400/60% saline), and alcohol (e.g., 40% ethanol/60% water). A preferred pharmaceutically acceptable carrier for use in conjunction with the present invention is polyethylene glycol, such as PEG 400, and particularly a composition comprising 40% PEG 400 and 60% water or saline.

The choice of carrier will be determined in part by the particular compound chosen, as well as by the particular method used to administer the comparison. Accordingly, there is a wide variety of suitable formulations of the pharmaceutical composition of the present invention.

The following formulations for oral, aerosol, parenteral, subcutaneous, intravenous, intraarterial, intramuscular, interperitoneal, rectal, and vaginal administration are merely exemplary and are in no way limiting.

The pharmaceutical compositions can be administered parenterally, e.g., intravenously, intraarterially, intrathecally, subcutaneously, intradermally, or intramuscularly. Thus, the invention provides compositions for parenteral administration that comprise a solution of the compound dissolved or suspended in an acceptable carrier suitable for parenteral administration, including aqueous and non-aqueous, isotonic sterile injection solutions.

Overall, the requirements for effective pharmaceutical carriers for parenteral compositions are well known to those of ordinary skill in the art. See Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986). Such solutions can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound may be administered in a physiologically acceptable diluent or a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxdlane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, allyl-β-aminopropionates, and 2-alkyl-imidazoline quaternary ammonium salts, and (e) combinations thereof.

The parenteral formulations typically will contain from about 0.5% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Topical formulations, including those that are useful for transdermal drug release, are well known to those of skill in the art and are suitable in the context of the present invention for application to skin.

Formulations suitable for oral administration require extra considerations considering the nature of some of the compounds of the present invention and the likely breakdown thereof if such compounds are administered orally without protecting them from the digestive secretions of the gastrointestinal tract. Such a formulation can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmelose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compounds of the present invention, alone or in combination with other suitable components, can be made into aerosol formulations to be administered via inhalation. The compounds are preferably supplied in finely divided form along with a surfactant and propellant. Typical percentages of active compound are 0.01-20% by weight, preferably 1% -10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compounds and polymers useful in the present inventive methods may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The concentration of the compounds of the present invention in the pharmaceutical formulations can vary widely, i.e., from less than about 1%, usually at or at least about 10%, to as much as 20% to 50% or more by weight, and will be selected primarily by fluid volumes, or viscosities, in accordance with the particular mode of administration selected.

Thus, a typical pharmaceutical composition for intravenous infusion could be made up to contain 250 ml of sterile Ringer's solution, and 100 mg of the compound. Actual methods for preparing parenterally administrable compounds will be known or apparent to those skilled in the art and are described in more detail in, for example, Remington's Pharmaceutical Science (17th ed., Mack Publishing Company, Easton, Pa., 1985).

It will be appreciated by one of ordinary skill in the art that, in addition to the afore described pharmaceutical compositions, the compounds of the present inventive method may be formulated as inclusion complexes, such as cyclodextrin inclusion complexes, or liposomes. Liposomes serve to target the compounds to a particular tissue, such as lymphoid tissue or cancerous hepatic cells. Liposomes can also be used to increase the half-life of the compound. Liposomes useful in the present invention include emulsions, foams, micelles, insoluble monolayers, liquid crystals, phospholipid dispersions, lanellar layers and the like. In these preparations, the compound to be delivered is incorporated as part of a liposome, alone or in conjunction with a suitable chemotherapeutic agent. Thus, liposomes filled with a desired compound of the invention can be directed to the site of a specific tissue type, hepatic cells, for example, where the liposomes then deliver the selected chemotherapeutic-enhancement compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871; 4,501,728; 4,837,028; and 5,019,369. For targeting to the cells of a particular tissue type, a ligand to be incorporated into the liposome can include, for example, antibodies or fragments thereof specific for cell surface determinants of the targeted tissue type. A liposome suspension containing a compound may be administered intravenously, locally, topically, etc. in a dose that varies according to the mode of administration, the compound being delivered, or the stage of disease being treated.

The present invention has applicability to the treatment of any type of cancer capable of being treated with an antineoplastic alkylating agent which causes cytotoxic lesions at the O⁶-position of guanine. Such cancers include, for example, colon tumors, prostrate tumors, brain tumors, lymphomas, leukemias, breast tumors, ovarian tumors, lung tumors, Wilms' tumor, rhabdomyosarcoma, multiplemyeloma, stomach tumors, soft-tissue sarcomas, Hodgkin's disease, and non-Hodgkin's lymphomas. In view of the mode of action of the compounds of the present invention, such compounds can be used in conjunction with any type of antineoplastic alkylating agent which causes cytotoxic lesions at the O⁶ -position of guanine. In an embodiment, the antineoplastic alkylating agent is a chloroethylating agent or a methylating agent. Thus, for example, the alkylating agent is selected from the group consisting of lomustine, carmustine, semustine, nimustine, fotomustine, mitozolomide, clomesone, temozolomide, dacarbazine, procarbazine, streptzocin, and combinations thereof. In accordance with the invention, antineoplastic alkylating agents include, for example, chloroethylating agents (e.g. chlioroethylnitrosoureas and chloroethyltriazines) and monofunctional alkylating agents such as streptozotocin, procarbazine, dacarbazine, and temozolomide.

Among the chloroethylating agents, the most frequently used chemotherapeutic drugs are 1-(2-chloroethyl)-3-cyclohexyl-1-nitrosourea (CCNU, lomustine), 1,3-bis(2-chloroethyl)-1-nitrosourea (BCNU, carmustine), 1-(2-chloroethyl)-3-(4-methylcyclohexyl)-1-nitrosourea (MeCCNU, semustine), and 1-(2-chloroethyl)-3-(4-amino-2-methyl-5-pyrimidinyl)methyl-1-nitrosourea (lomustine, ACNU). These agents have been used clinically against tumors of the central nervous system, multiple myeloma, melanoma, lymphoma, gastrointestinal tumors, and other solid tumors (Colvin and Chabner, Alkylating Agents. In: Cancer Chemotherapy: Principles and Practice, Chabner and Collins, eds., Lippincott, Philadelphia, pp. 276-313 (1990); McCormick and McElhinney, Eur. J. Cancer, 26, 207-221 (1990)). Chloroethylating agents currently under development with fewer side effects are 1-(2-chloroethyl)-3-(2-hydroxyethyl)-1-nitrosourea (HECNU), 2-chloroethyl-methylsulfonylmethanesulfonate (clomesone), and 1-[N-(2-chloroethyl)-N-nitrosoureido]ethylphosphonic acid diethyl ester (fotomustine) (Colvin and Chabner, Alkylating Agents. In: Cancer Chemotherapy: Principles and Practice, Chabner and Collins, eds., Lippincott, Philadelphia, pp. 276-313 (1990); McCormick and McElhinney, Eur. J. Cancer, 26, 207-221 (1990)). Methylating chemotherapeutic agents include streptozotocin (2-deoxy-2-(3-methyl-3-nitrosoureido)-D-glucopyranose), procarbazine (N-(1-methylethyl)-4-[(2-methylhydrazino)methyl]benzamide), dacarbazine or DTIC (5-(3,3-dimethyl-1-triazenyl) -1H-imidazole-4-carboxamide), and temozolomide (8-carbamoyl-3-methylimidazol[5,1 d]-1,2,3,5-tetrazine-4-(3H)-one). Temozolomide is active against malignant melanomas, brain tumors, and mycosis fungoides. Streptozotocin is effective against pancreatic tumors. Procarbazine is used to treat Hodgkin's disease and brain tumors, and DTIC is used in treatment of melanoma and lymphomas (Colvin and Cabner, Alkylating Agents. In: Cancer Chemotherapy: Principles and Practice, Chabner and Collins, eds., Lippincott, Philadelphia, pp. 276-313 (1990); Longo, Semin. Concol., 17, 716-735 (1990)). The pharmaceutical composition of the present invention can include an antineoplastic alkylating agent.

Certain of the compounds of the present invention have selectivity for certain types of tumor cells. In an embodiment, the tumor cells to be treated by the compounds of the present invention express a folate receptor; particularly the α-folate receptor. In accordance with an embodiment of the present invention, the tumor cells are selected from the group consisting of nasopharyngeal carcinomas, adenocarcinomas, ovarian carcinomas, endometrial carcinomas, bronchioloalveolar carcinomas, non-small cell lung carcinomas, small cell lung carcinomas, squamous carcinomas, colorectal carcinomas, gastric carcinomas, and kidney carcinomas.

The present invention further provides a method of inhibiting the reaction of AGT with an alkylated DNA comprising reacting the AGT with a compound of formula (I): 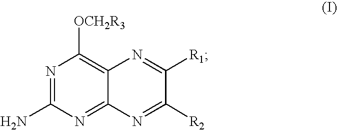
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxyalkyl wherein the alkyl is C₁-C₆, halo, hydroxy, aryl, amino, monoalkylamino wherein the alkyl is C₁-C₆, dialkylamino wherein the alkyl is-C₁-C₆, acylamino, C₁-C₆ alkyl substituted aryl, nitro, C₃-C8 cycloalkyl, C₂-C₆ alkenyl, C₂-C₆ alkynyl, and a group of formula (II): 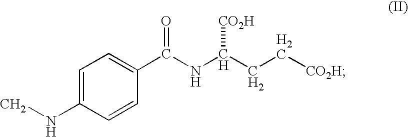
R₃ is (a) phenyl or (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, the cyclic group optionally with a carbocyclic or heterocyclic ring fused thereto, which is substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, Cl -C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof, with the proviso that R₁ and R₂ are not simultaneously hydrogen. The reaction can be in vitro or in vivo.

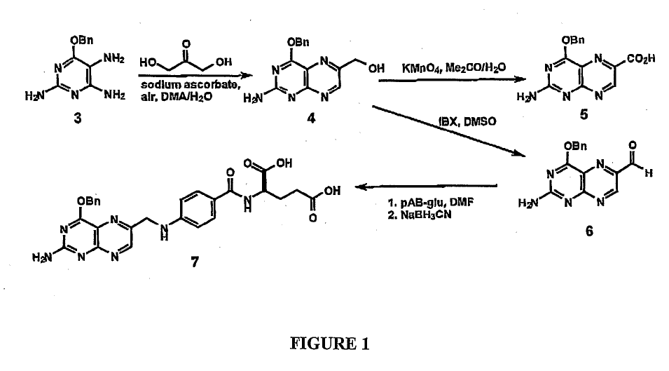

The compounds of the present invention can be prepared by any suitable method. For example, compounds wherein R₃ is phenyl can be prepared as follows. Pteridines 1 (R₁, R₂=H, R₃=phenyl) and 2 (R₁, R₂=CH₃, R₃=phenyl) were prepared by the method of Pfleiderer and Lohrmann from 2,4,5-triamino-O⁶-benzylpyrimidine (3) (FIG. 1) and glyoxal or diacetyl, respectively.¹¹ Pteridines 4-7 were prepared as illustrated in FIG. 1. Thus, treatment of the triaminopyrimidine (3) with dihydroxyacetone in dimethylacetamide (DMA)/H₂O (1:1) in the presence of sodium ascorbate and air afforded the hydroxymethylpteridine (4), which was oxidized to the 6-carboxyl derivative (5) with permanganate in acetone water solution. Alternatively, 4 was oxidized to the 6-formylpteridine derivative 6 by treatment with iodoxybenzoic acid (IBX) in dimethylsulfoxide. Treatment of 6 with p-aminobenzoylglutamate (pAB-glu) followed by reduction with sodium cyanoborohydride in dimethylformamide led to formation of O⁴-benzylfolic acid (7).

Compounds of the present invention wherein R₃ is a heterocyclic ring can be prepared by methods known to those skilled in the art; see, for example, U.S. Pat. No. 6,096,724.

The ability of these various compounds to inactivate the human alkyltransferase protein in the presence and absence of calf thymus DNA is summarized Table 1. Data for O⁶-benzylguanine are included for comparison. The data are expressed as concentration of inactivator required to reduce the activity of the alkyltransferase protein by 50% (i.e. ED₅₀). As indicated, in the absence of calf thymus DNA, pteridine derivatives 2 and 4 exhibit activity similar to that of O⁶-benzylguanine although derivatives 1 and 5-7 are superior to O⁶-benzylguanine as alkyltransferase inactivators. In particular, O⁴-benzylfolic acid (7) is roughly thirty times more effective than O⁶-benzylguanine against the wild-type human alkyltransferase and displays an ED₅₀ in the nM range. Interestingly, this same compound is also effective against the P140K mutant alkyltransferase (although at significantly higher concentrations). This protein is essentially resistant to inactivation by O⁶-benzylguanine and related derivatives.¹⁰ Previously, only oligodeoxyribonucleotides containing O⁶-benzylguanine residues were known to inactivate the P140K and other mutant alkyltransferase proteins.¹⁰ In the presence of calf thymus DNA, the ED₅₀ values for alkyltransferase inactivation increase significantly suggesting that DNA binding of the protein hinders access of these 2-amino-O⁴-benzylpteridines to the protein's active site. This contrasts with the situation for O⁶-benzylguanine (Table 1) which exhibits an enhanced ED₅₀ in the presence of calf thymus DNA.¹² Nevertheless, all the derivatives 1, 2 and 4-7 exhibit significant alkyltransferase inactivating ability even in the presence of calf thymus DNA and could therefore be useful against the DNA bound form of the protein as well. Although O⁴-benzylfolic acid was capable of inactivating the P140K mutant in the absence of calf thymus DNA, it is inactive against this protein at concentrations as high as 1 mM in the presence of calf thymus DNA. DNA clearly prevents access of 7 to the mutant protein's active site.

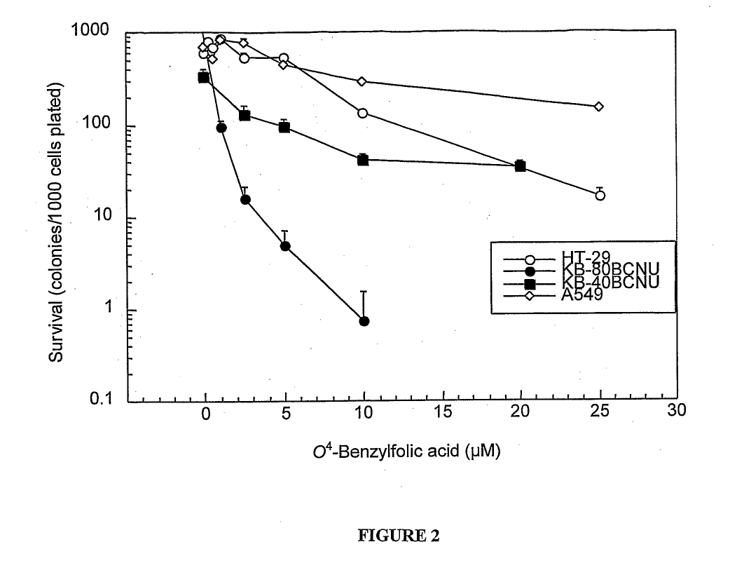

The 2-amino-O⁴-benzylpteridine derivatives 1, 2, 4, 6 and 7 are all capable of enhancing HT 29 cell killing by BCNU (Table 2). 2-Amino-O⁴-benzyl-6-carboxypteridine (5) is not effective probably because the negative charge on the molecule at physiological pH prevents its easy entry into cells. However, even though O⁴-benzylfolic acid is also anionic at physiological pH, it does enhance cell killing by BCNU. Furthermore, its effectiveness as an adjuvant is a function of the cells' ability to express the α-form of the folic acid receptor. This is illustrated in FIG. 2 which shows A549 lung tumor, HT29 colon tumor and KB nasopharyngeal tumor cell killing by 40 μM BCNU following a two hour exposure to 7 in folate free growth medium. KB cell killing by 80 μM BCNU in combination with 7 is also illustrated. For these experiments (FIG. 2) cells were grown in RPMI medium, were incubated with 7 for 2 hr and were then treated with either 40 μM BCNU (A549, HT29 and KB cells) or 80 μM BCNU (KB cells) for 2 hr. The medium was then replaced with fresh medium containing no 7. Cells were kept for 16-18 hr before being replated. As shown, the effectiveness of 7 as an adjuvant was lowest in A549 cells, was somewhat greater in HT29 cells, and was greatest in KB cells. A549 cells express little, if any oc folate receptor, HT29 cells express low levels of the receptor, and KB cells express high levels of the receptor.₁₄₋₁₇ Thus, O⁴-benzylfolic acid may be a useful agent for selectively inactivating alkyltransferase in tumors that over-express the α folate receptor. These tumors are numerous and include adenocarcinomas; ovarian, endometrial and bionchioloalveolar carcinomas; some non-small cell lung carcinomas, small cell lung carcinomas, squamous cell carcinomas, colorectal carcinomas, gastric carcinomas and kidney tumors.¹⁷ Such tumor selectivity would be very advantageous since the side effects associated with systemic alkyltransferase inactivation^{7,8} could be significantly reduced.

TABLE 1
Inactivation of human O⁶-alkylguanine-DNA alkyltransferase in vitro
in the absence and presence of calf thymus (ct) DNA

| | −ctDNA | | +ctDNA | |
|---|---|---|---|---|
| Inactivator | ED₅₀ (μM) | (n) | ED₅₀ (μM) | (n) |
| O⁶-benzylguanine | 0.32 ± 0.08 | (4) | 0.12 ± 0.02 | (3) |
| 2-amino-O⁴-benzylpteridine (1) | 0.045 ± 0.01 | (4) | 0.45 ± 0.05 | (6) |
| 2-amino-O⁴-benzyl-6,7- | 0.4 | (1) | 0.5 | (1) |
| dimethylpteridine (2) | | | | |
| 2-amino-O⁴-benzyl-6- | 0.2 | (1) | 0.4 | (1) |
| hydroxymethylpteridine (4) | | | | |
| 2-amino-O⁴-benzyl-6- | 0.09 | (2) | 1.83 ± 0.62 | (3) |
| carboxypteridine (5) | | | | |
| 2-amino-O⁴-benzyl-6- | 0.19 ± 0.01 | (2) | 1.05 ± 0.3 | (2) |
| formylpteridine (6) | | | | |
| O⁴-benzylfolic acid (7)^{a} | 0.01 ± 0.001 | (3) | 0.47 ± 0.05 | (3) |

^{a}ED₅₀ against the P140K mutant alkyltransferase in the absence of ctDNA = 12 μM. In the presence of ctDNA the compound is inactive at concentrations ≦1 mM.

TABLE 2
Concentration of 2-amino-O⁴-benzylpteridine derivatives
required to kill 90% of HT29 cells (ED₉₀) with BCNU (40 μM).^{a}

| | ED₉₀ (μM) | |
|---|---|---|
| | Culture Medium | |
| Inactivator | Dulbecco's | RPMI |
| O⁶-benzylguanine | 0.4^{b} | 0.7^{c} |
| 2-amino-O⁴-benzylpteridine (1) | 0.2^{b} | |
| 2-amino-O⁴-benzyl-6,7-dimethylpteridine (2) | 0.6^{b} | |
| 2-amino-O⁴-benzyl-6-hydroxymethylpteridine (4) | 0.7^{b} | |
| 2-amino-O⁴-benzyl-6-carboxypteridine (5) | inactive | |
| | at 30 μM^{b} | |
| 2-amino-O⁴-benzyl-6-formylpteridine (6) | | 0.7^{c} |
| O⁴-benzylfolic acid (7) | | 24^{c,d} |

^{a}Cells were completely resistant to 40 μM BCNU treatment in the absence of alkyltransferase inactivator. ^{b}Alkyltransferase inactivator was present before, during and after treatment with BCNU for 16-18 hours before replating (see Experimental Section). ^{c}Alkyltransferase inactivator was present before and during BCNU treatment only. ^{d}ED₉₀=15 μM in RPMI folate-free medium.

The following example further illustrates the invention but, of course, should not be construed as in any way limiting its scope.

### EXAMPLE

This example demonstrates a method of preparing some of the compounds of the present invention.

Materials and Methods. Unless otherwise stated, chemicals were obtained from Aldrich, Milwaukee, Wis. or Sigma, St. Louis, Mo. and were used without further purification. UV spectra were determined on a Beckman Coulter DU 7400 spectrophotometer. ¹H and ¹³C NMR spectra were recorded in DMSO-d₆ with a Varian INOVA 400 MHz spectrometer. Chemical shifts are reported as δ values in parts per million relative to TMS as internal standard. The splitting pattern abbreviations are as follows: s=singlet, d=doublet, dd=double doublet, t=triplet, m=multiplet. Elemental analyses, performed by Atlantic Microlab, Inc., Norcross, Ga. were within 0.4% of the theoretical values calculated for C, H, and N. Thin layer chromatographic analyses were performed using precoated, aluminum-backed silica gel plates and the spots were visualized with UV light. All silica gel column chromatography was carried out using Davisil grade 633, 200-425 mesh, 60◯. Compounds 1, 2, and 3 were prepared by the method of Pfleiderer and Lolinnann.¹¹

2-Amino-O⁴-benzyl-6-hydroxymethylpteridine (4). 2,4,5-Triamino-O⁶-benzylpyrimidine (3) (3.26 g, 14.1 mmol) was dissolved in DMA/H₂O (1:1, 28 mL) and stirred at room temperature. Sodium ascorbate (2.85 g, 14.4 inmol) was added, followed by dihydroxyacetone dimer (2.57 g, 14.3 mmol). The reaction mixture was stirred at 40° C., and air was bubbled into the reaction flask through a Pasteur pipet. The reaction was monitored by TLC (10:1 CH₂Cl₂:MeOH). After 4 hr all the starting material was consumed, and the reaction mixture was poured into H₂O (250 mL) producing a yellow-orange solid. The yellow-orange solid was collected by filtration, was dissolved in CH₂Cl₂:MeOH (3:1, 500 mL) and was dried over MgSO₄. The solution was filtered and evaporated onto silica gel (100 mL). Product was eluted from a silica gel column with CH₂Cl₂:MeOH (20:1) and fractions containing product were pooled and-evaporated to produce 2-amino-O⁴-benzyl-6-hydroxymethylpteridine (4) (1.12 g, 28.1%). UV (MeOH/0.05M phosphate, pH=6.8, 5:95) λₘₐₓ 234 nm (ε=18800), 264 nm (ε=9200), 366 (ε=6900); ¹H NMR δ 8.88 (1H, s, H-7), 7.56 (2H, m, ArH), 7.40 (3H, m, ArH), 7.28 (2H, s, N²H₂, exchange with D₂O), 5.58 (1H, t, J =5.9 Hz, 6-CH₂OH, exchanges with D₂O), 5.55 (2H, s, ArCH₂), 4.62 (2H, d, J=5.9 Hz, 6-CH₂OH changes to a singlet in D₂O); ¹³C NMR (100 MHz) δ 166.4, 161.2, 156.5, 151.1, 150.0, 135.8, 128.9, 128.5, 128.3, 121.2, 68.4, 62.7; Anal. Calcd. for C₁₄H₁₃N₅O₂.0.5H₂O: C, 57.53; H, 4.83; N, 23.96. Found: C, 57.52; H, 4.72; N, 23.76.

2-Amino-O⁴-benzylpteridine-6-carboxylic acid (5). 2-Amino-O⁴-benzyl-6-hydroxymethylpteridine (4) (0.24 g, 0.84 mmol) was suspended in acetone:0.5 M phosphate buffer, pH=7 (1:1, 14mL) and stirred at room temperature. Potassium permanganate (0.34 g, 2.18 mmol) was added in 4 portions at 30 minute intervals. The resulting suspension was then stirred at room temperature for an additional 3 hours. The reaction mixture was diluted with H₂O (50 mL). Sodium sulfite was added until all of the permanganate was consumed, producing a brown-black precipitate, which was removed by filtration, leaving a clear, yellow solution. The pH was adjusted to 2.5 by the addition of 2 M HCl producing a yellow solid, which was collected by filtration. The solid was dissolved in H₂O (50 mL) by adjusting the pH to 7.0 through the addition of 0.1 M NaOH until the pH remained constant for 30 minutes. Any suspended solid material was filtered and the solution was evaporated to give the sodium salt. This product was purified on a 3×80 cm Sephadex LH-20 column eluted with H₂O (1 mL/min). UW absorption was monitored continuously at 280 nm. Fractions (10 mL) 34-44 containing the product were combined, the pH was adjusted to 2.5 with HCl to precipitate the product, which was collected by filtration, and dried under vacuum to afford (5) (0.17 g, 67%). ¹H NMR δ 13.52 (1H, S, CO₂H, exchanges with D₂O), 9.25 (1H, s, H-7), 7.83 (1H, N²Hₐ, exchanges with D₂O), 7.70 (1H, N²H_{b}, exchanges with D₂O), 7.58 (2H, m, ArH), 7.41 (3H, m, ArH), 5.60 (2H, s, ArCH₂); ¹³C NMR (100 MHz) δ 166.9, 164.8, 162.6, 158.1, 151.3, 137.4, 135.5, 129.0, 128.5, 128.45, 122.5, 68.75; Anal. Calcd. for C₁₄H₁₁N₅O₃.H₂O: C, 53.33; H, 4.16; N, 22.21. Found: C, 53.67; H, 3.98; N, 22.40.

2-Amino-O⁴-benzyl-6-formylpteridine (6). Iodoxybenzoic acid (IBX) (1.7 g, 6.1 mmol) was stirred in DMSO (16 mL) until dissolved. 2-Amino-O⁴-benzyl-6-hydroxymethylpteridine (4) (1.16 g, 4.1 mmol) was added with constant stirring at room temperature to produce a dark orange solution. The reaction was complete in 2 hr as monitored by TLC (CH₂Cl₂:MeOH, 10:1). The reaction mixture was poured into H₂O (150 mL) to produce a pale yellow precipitate, which was collected by filtration. This solid was stirred at 40° C. in CH₂Cl₂:acetone (1:1, 250 mL) for approximately 30 min and was filtered to remove the iodosobenzoic acid byproduct. This process was repeated twice. The dissolved product was evaporated onto silica (50 mL) and was eluted from a silica gel column with CH₂Cl₂:CH₃CN (7:3). Solvent was evaporated to give 2-amino-O⁴-benzyl-6-formylpteridine (6) (0.26 g, 0.92 mmol, 22.4%). UV (MeOH/0.05 M phosphate, pH=6.8, 5:95) λₘₐₓ 236 nm (ε=13600), 261 (sh) (ε=9600) 309 nm (ε=5200), 370 (ε=9300); ¹H NMR δ 9.96 (1H, s, 6-CHO) 9.19 (1H, s, H-7), 8.03 (1H, s, N²Hₐ, exchanges with D₂O), 7.89 (1H, s, N²H_{b}, exchanges with D₂O) 7.60 (2H, m, ArH), 7.42 (3H, m, ArH), 5.62 (2H, s, ArCH₂); ¹³C NMR (100 MHz) δ 191.2, 166.9, 163.1, 159.0, 149.2, 141.0, 135.4, 129.1, 128.51, 128.49, 122.8, 69.0; Anal. Calcd. for C₁₄H₁₁N₅O₂: C, 59.78; H, 3.94; N, 24.90. Found: C, 59.80; H, 4.03; N, 24.86.

O⁴-Benzylfolic acid (7). 2-Amino-O⁴-benzyl-6-formylpteridine (6) (0.26 g, 0.92 mmol) and p-aminobenzoylglutamate (pAB-glu) (0.29 g, 1.1 mmol) were stirred in DMF (4.4 mL) until completely dissolved. Acetic acid (0.04 mL) was added, followed by NaBH₃CN (0.08 g, 1.3 mmol). After approximately 5 min, the reaction color changed from yellow-orange to red. TLC (CH₂Cl₂:MeOH:AcOH, 90:5:5) showed complete loss of 6. The reaction mixture was poured into vigorously stirred water (50 mL), producing a yellow precipitate that dissolved when the pH of the suspension was adjusted to 7.2 by the addition of 2 M NaOH. Activated charcoal (20 mg) was added and was filtered. The solution pH was then adjusted to 3.0 by the addition of 2 M HCl , producing a yellow precipitate, which was collected by filtration. The solid was dissolved in CH₂Cl₂:MeOH (3:1) and evaporated onto silica (30 mL). The product was eluted from a silica gel column with CH₂Cl₂:MeOH:AcOH (90:5:5). The solvent was evaporated and the product was suspended in vigorously stirred H₂O to produce a fine solid (7) which was filtered and dried under vacuum (87 mg, 0.16 mmol, 17.7%). UV (0.05 M phosphate, pH=6.8) λₘₐₓ 277 nm (ε=19000), 289 nm (sh) (ε=18100) 368 nm (ε=8200); ¹H NMR δ 12.31 (2H, br-s, 2CO₂H), 8.79 (1H, s, 7-H), 8.12 (1H, d, J=7.7 Hz, glu-NH, exchanges with D₂O), 7.64 (2H, d, J=8.7 Hz pABArH), 7.57 (2H, m, BnArH), 7.41 (3H, m, BnArH), 7.29 (2H, br-s, N²H₂, exchange with D₂O), 6.95 (1H, t, J=6.1 Hz, 6-CH₂NH, exchanges with D₂O), 6.64 (2H, d, J=8.8 Hz, pABArH), 5.58 (2H, s, BnCH₂), 4.50 (2H, d, J=5.7 Hz, 6-CH₂NH, singlet in D₂O), 4.33 (1H, m, gluα-CH, dd in D₂O), 2.32 (2H, t, J=7.5 Hz gluγ-CH₂), 2.04(1H, m, gluβ-CH₂ₐ), 1.90 (1H, m, gluβ-CH_{2b}); ¹³C NMR (100 MHz) δ 173.9, 173.7, 166.4, 166.3, 161.3, 156.5, 150.8, 150.3, 149.1, 135.9, 129.0, 128.8, 128.5, 128.3, 121.6, 121.3, 111.2, 68.4, 51.7, 46.1, 30.4, 24.0; Anal. Calcd. for C₂₆H₂₅N₇O₆.0.5H₂O: C, 57.77; H, 4.85; N, 18.14. Found: C, 57.72; H, 4.71; N, 18.29. Alternatively, the crude product before silica gel chromatography (see above) was dissolved in H₂O, the pH of which was adjusted to 7.0 by the addition of 2 M NaOH, and the solution was evaporated to give the sodium salt. This product was purified on a Sephadex LH-20 column (3×80 cm), eluted in pure H₂O (1 mL/min). UV absorption was monitored continuously at 280 nm. Fractions (10 mL) 24-33 containing the product were combined, the pH was adjusted to 2.5 with HCl to precipitate the product, which was collected by filtration and dried under vacuum.

In Vitro AGT Activity Assay

Purified histag-hAGT was incubated with different concentrations of inactivator in 0.5 mL of reaction buffer (50 mM Tris-HCL, pH 7.6, 0.1 mM EDTA, 5.0 mM dithiothreitol) containing 50 μg hemocyanin for 30 min at 37° C. The remaining AGT. activity was determined after incubation with [³H]methylated calf thymus DNA substrate for 30 min at 37° C. by measuring the [³H]methylated protein formed, which was collected on nitrocellulose filters. ¹⁸ The results were expressed as the percentage of the AGT activity remaining. The concentration of inhibitor which led to a 50% loss of AGT activity (ED₅₀) was calculated from graphs of the percentage of remaining AGT activity against inactivator concentration. For assays in the presence of DNA, 10 μg of calf thymus DNA was added before incubation with the inactivators.

Cell Culture and Cytotoxicity Assays

Cells were grown either in Dulbecco's modified Eagle's medium supplemented with 10% fetal bovine serum plus 1.5 mM glutamine and 50 μg/mL gentamycin (HT29) or in RPMI 1640 medium in the presence of 10% fetal bovine serum (HT29, A549 and KB cells). The effect of AGT inactivators on the sensitivity of cells to BCNU was determined using a colony-forming assay.¹³ Cells were plated at a density of 10⁶ in 25-cm² flasks and 24 h later were incubated with different concentrations of AGT inactivator for 2 h before exposure to 40 μM (HT29 cells and A549 cells) or 80 μM (KB cells) of BCNU for 2 h. The BCNU was first dissolved in absolute ethanol at a concentration of 8 mM, was diluted with the same volume of ice-cold phosphate-buffered saline and was immediately used to treat the cells. The medium was replaced with fresh medium containing AGT inactivator (where indicated) and the cells were left to grow for an additional 16-18 h. The AGT inhibitor was added to the medium after the treatrment with BCNU to ensure that the inhibitor was present during the entire period that DNA adducts are formed by BCNU. The cells were then replated at densities of 200-2000 cells/25-cm² flasks and grown for 8 days until discrete colonies were formed. The colonies were washed with 0.9% saline solution, were stained with 0.5% crystal violet in ethanol, and counted. The plating efficiency of cells not treated with drugs was about 50% for HT29 and A549 cells and 80% for KB cells. In experiments to assess the effect of folate present in the medium on the sensitivity of cells to the AGT inhibitors and BCNU, the cells were incubated with drugs for 2 h and BCNU for 2 h in RPMI 1640 folate-free medium. After this period, the medium was replaced with fresh RPMI 1640 medium.

### REFERENCES

(1) Moschel, R. C.; McDougall, M. G.; Dolan, M. E.; Stine, L.; Pegg, A. E. Structural featues of substituted purine derivatives compatible with depletion of human O⁶-alkylguanine-DNA alkyltransferase. J Med. Chem. 1992, 35, 4486-4491.
(2) Chae, M.-Y.; McDougall, M. G.; Dolan, M. E.; Swenn, K.; Pegg, A. E.; Moschel, R. C. Substituted O⁶-benzylguanine derivatives and their inactivation of human O⁶-alkylguanine-DNA alkyltransferase. J Med. Chem. 1994, 37, 342-347.
(3) Chae, M.-Y., Swenn, K.; Kanugula, S.; Dolan, M. E.; Pegg, A. E.; Moschel, R. C. 8-Substituted O⁶-benzylguanine, substituted 6(4)-(benzyloxy)pyrimidine, and related derivatives as inactivators of human O⁶-alkylguanine-DNA alkyltransferase. J Med. Chem. 1995, 38, 359-365.
(4) McElhinney, R. S.; Donnelly, D. J.; McCormick, J. E.; Kelly, J.; Watson, A. J.; Rafferty, J. A.; Elder, R. H.; Middleton, M. R.; Willington, M. A.; McMurry, T. B. H.; Margison, G. P. Inactivation of O⁶-alkylguanine-DNA alkyltransferase. 1. Novel O⁶-(heteromethyl)guanines having basic rings in the side chain. J Med. Chem. 1998, 41, 5265-5271.
(5) Griffin, R. J.; Arris, C. E.; Bleasdale, C.; Boyle, F. T.; Calvert, A. H.; Curtin, N. J.; Dalby, C.; Kanugula, S.; Lembicz, N. K.; Newell, D. R.; Pegg, A. E.; Golding, B. T. Resistance-modifying agents. 8. Inhibition of O⁶-alkylguanine-DNA alkyltransferase by O⁶-alkenyl-, O⁶-cycloalkenyl-, and O⁶-(2-oxoalkyl)guanines and potentiation of temozolomide cytotoxicity in vitro by O⁶-(1-cyclopentenylmethyl)guanine. J Med. Chem. 2000, 43, 4071-4083.
(6) Pegg, A. E.; Dolan, M. E.; Moschel, R. C. Structure, function and inhibition of O⁶-alkylguanine-DNA alkyltransferase. Prog. Nucleic Acid Res. Mol. Biol. 1995, 51, 167-223.
(7) Friedman, H. S.; Koklinalis, D. M.; Pluda, J.; Friedman, A. H.; Cokgor, I.; Haglund, M. M.; Ashley, D. M.; Rich, J.; Dolan, M. E.; Pegg, A. E.; Moschel, R. C.; McLendon, R. E.; Tracy, K.; Herndon, J. E.; Bigner, D. D.; Schold, S. C. Phase I trial of O⁶-benzylguanine for patients undergoing surgery for malignant glioma. J Clin. Oncol. 1998, 16, 3570-3575.
(8) Quinn, J. A.; Pluda, J.; Dolan, M. E.; Delaney, S.; Kaplan, R.; Rich, J. N.; Friedman, A. H.; Reardon, D. A.; Sampson, J. H.; Colvin, O. M.; Haglund, M. M.; Pegg, A. E.; Moschel, R. C.; McLendon, R. E.; Provenzale, J. M.; Gururangan, S.; Tourt-Uhlig, S.; Herndon, J. E.; Bigner, D. D.; Friedman, H. S. Phase II trial of carmustine plus O⁶-benzylguanine for patients with nitrosourea-resistant recurrent or progressive malignant glioma. J. Clin. Oncol. 2002, 20, 2277-2283.
(9) Middleton, M. R.; Margison, G. P. Improvement of chemotherapy efficacy by inactivation of a DNA-repair pathway. Lancet Oncol. 2003, 4, 37-44.
(10) Luu, K. X.; Kanugula, S.; Pegg, A. E.; Pauly, G. T.; Moschel, R. C. Repair of oligodeoxyribonucleotides by O⁶-alkylguanine-DNA alkyltransferase. Biochemistry 2002, 41, 8689-8697.
(11) Pfleiderer, W.; Lohrmann, R. Synthese von 2-amino-4-alkoxy-pteridinen. Chem. Ber. 1961, 94, 12-18.
(12) Pegg, A. E.; Chung, L.; Moschel, R. C. Effect of DNA on the inactivation of O⁶-alkylguanine-DNA alkyltransferase by 9-substituted O⁶-benzylguanine derivatives. Biochem. Pharmacol. 1997, 53, 1559-1564.
(13) Dolan, M. E.; Moschel, R. C.; Pegg, A. E. Depletion of mammalian O⁶-alkylguanine-DNA alkyltransferase activity by O⁶-benzylguanine provides a means to evaluate the role of this protein in protection against carcinogenic and therapeutic alkylating agents. Proc. Natl. Acad. Sci. USA, 1991, 87, 5368-5372.
(14) Ross, J. F.; Chaudhuri, P. K.; Ratnam, M. Differential regulation of folate receptor isoforms in normal and malignant tissues in vivo and in established cell lines. Physiologic and clinical implications. Cancer, 1994, 73, 2432-2443.
(15) Nonancourt-Didion, M. d.; Gueant, J.-L.; Adjalla, C.; Chery, C.; Hatier, R.; Namour, F. Overexpression of folate binding protein α is one of the mechanism explaining the adaptation of HT29 cells to high concentration of methotrexate. Cancer Lett. 2001, 171, 139-145.
(16) Wang, S.; Luo, J.; Lantrip, D. A.; Waters, D. J.; Mathias, C. J.; Green, M. A.; Fuchs, P. L.; Low, P. S. Design and synthesis of [¹¹¹In]DTPA-folate for use as a tumor-targeted radiopharmaceutical. Bioconjugate Chemn. 1997, 8, 673-679.
(17) Ladino, C. A.; Chari, R. V. J.; Bourret, L. A.; Kedersha, N. L.; Goldmacher, V. S. Folate-maytansinoids: target-selective drugs of low molecular weight. Int. J Cancer, 1997, 73, 859-864.
(18) Kanugula, S.; Pegg, A. E. Novel DNA repair alkyltransferase from Caenorhabditis elegans. Environ. Mol. Mutagenesis, 2001, 38, 235-243.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 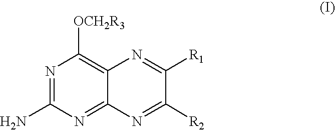
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl, and a group of formula (II): 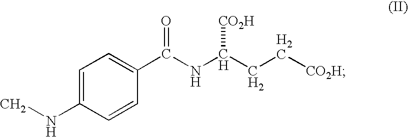
R₃ is (a) phenyl; (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, said cyclic group optionally with a carbocyclic or heterocyclic ring fused thereto, which is substituted with 1 to 5 substituents selected from the group consisting of halogen, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl;
or a pharmaceutically acceptable salt thereof;
with the provisos that (1) R₁ and R₂ are not simultaneously hydrogen; and (2) when R₃ is unsubstituted phenyl, R₁ and R₂ are not simultaneously methyl.

2. The compound of claim 1, wherein R₃ is phenyl or a phenyl group substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof.

3. The compound of claim 2, wherein R₁ is selected from the group consisting of hydrogen, C₁-C₆ alkyl, carboxyl, formyl, C₁-C₆ hydroxyalkyl, C₁-C₆ carboxyalkyl, C₁-C₆ formyl alkyl, and a group of formula (II) and R₂ is hydrogen or C₁-C₆ alkyl; and R₃ is phenyl; or a pharmaceutically acceptable salt thereof.

4. The compound of claim 3, wherein R₁ is selected from the group consisting of hydrogen, C₁-C₆ alkyl, C₁-C₆ hydroxyalkyl, carboxyl, formyl, and a group of formula (II) and R₂ is hydrogen or C₁-C₆ alkyl; or a pharmaceutically acceptable salt thereof.

5. The compound of claim 4, wherein R₁ is hydroxymethyl, carboxyl, formyl, or a group of formula (II) and R₂ is hydrogen; or a pharmaceutically acceptable salt thereof.

6. The compound of claim 5, wherein R₁ is hydroxymethyl; or a pharmaceutically acceptable salt thereof.

7. The compound of claim 5, wherein R₁ is carboxyl; or a pharmaceutically acceptable salt thereof.

8. The compound of claim 5, wherein R₁ is formyl; or a pharmaceutically acceptable salt thereof.

9. The compound of claim 5, wherein R₁ is a group of formula (II); or a pharmaceutically acceptable salt thereof.

10. A pharmaceutical composition comprising a pharmaceutically acceptable carrier and a compound or salt of claim 1.

11. The pharmaceutical composition of claim 10, further including an antineoplastic alkylating agent.

12. The pharmaceutical composition of claim 10, wherein the pharmaceutically acceptable carrier is polyethylene glycol.

13. The pharmaceutical composition of claim 11, wherein the antineoplastic alkylating agent is a chloroethylating agent.

14. The pharmaceutical composition of claim 11, wherein the antineoplastic alkylating agent is a methylating agent.

15. The pharmaceutical composition of claim 11, wherein the antineoplastic alkylating agent is selected from the group consisting of lomustine, carmustine, semustine, nimustine, fotomustine, mitozolomide, clomesone, temozolomide, dacarbazine, procarbazine, streptzocin, and combinations thereof.

16. A method of enhancing the chemotherapeutic treatment of tumor cells in a mammal with an antineoplastic alkylating agent that causes cytotoxic lesions at the O⁶-position of guanine, which method comprises administering to the mammal an effective amount of a compound of formula (I): 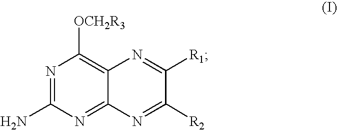
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl and a group of formula (II): 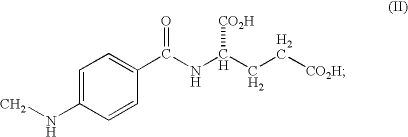
R₃ is (a) phenyl; (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, said cyclic group optionally with a carbocyclic or heterocyclic ring fuised thereto, which is substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl;
or a pharmaceutically acceptable salt thereof;
with the proviso that R₁ and R₂ are not simultaneously hydrogen;
and administering to the mammal an effective amount of an antineoplastic alkylating agent which causes cytotoxic lesions at the O⁶-position of guanine.

17. The method of claim 16, wherein R₃ is phenyl or a phenyl group substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₃-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′wherein n=0, 1, 2 or 3, R′ is H, a C₁-C₆ alkyl or aryl; or a pharmaceutically acceptable salt thereof.

18.-30. (canceled)

31. A method for treating tumor cells in a mammal comprising administering to the mammal an amount effective to reduce the O⁶-alkylguanine-DNA alkyltransferase activity in the mammal of a compound of formula (I): 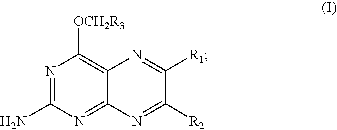
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxy C₁-C₆ alkyl, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl, and a group of formula (II): 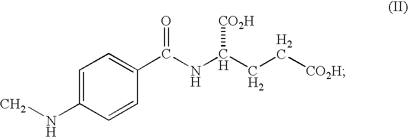
R₃ is (a) phenyl or (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, said cyclic group optionally with a carbocyclic or heterocyclic ring fused thereto, which is substituted with 1 to 5 substituents selected from the group consisting of halogen, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof,
with the proviso that R₁ and R₂ are not simultaneously hydrogen;
and administering to the mammal an effective amount of an antineoplastic alkylating agent which causes cytotoxic lesions at the O⁶-position of guanine.

32. The method of claim 31, wherein R₃ is phenyl or a phenyl group substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof.

33.-39. (canceled)

40. A method of inhibiting the reaction of O⁶-alkylguanine-DNA-alkyltransferase with an alkylated DNA comprising reacting the O⁶-alkylguanine-DNA-alkyltransferase with the compound of formula (I): 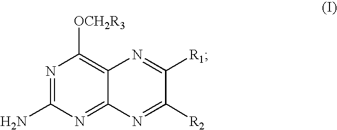
wherein R₁ and R₂ are independently selected from the group consisting of hydrogen, C₁-C₆ alkyl, carboxyl, formyl, C₁-C₆ hydroxyalkyl, C₁-C₆ carboxyalkyl, C₁-C₆ formyl alkyl, C₁-C₆ alkoxy, acyloxy, acyloxyalkyl wherein the alkyl is C₁-C₆, halo, hydroxy, aryl, amino, monoalkylamino wherein the alkyl is C₁-C₆, dialkylamino wherein the alkyl is C₁-C₆, acylamino, C₁-C₆ alkyl substituted aryl, nitro, C₃-C₈ cycloalkyl, C₂-C₆ alkenyl, C₂-C₆ alkynyl, and a group of formula (II): 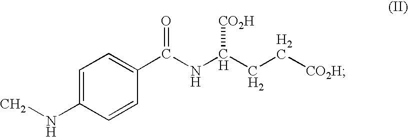
R₃ is (a) phenyl or (b) a cyclic group having at least one 5 or 6-membered heterocyclic ring, optionally with a carbocyclic or heterocyclic ring fused thereto, wherein each heterocyclic ring has at least one hetero atom chosen from O, N, or S; or (c) a phenyl group or a cyclic group, said cyclic group optionally with a carbocyclic or heterocyclic ring fused thereto, which is substituted with 1 to 5 substituents selected from the group consisting of halogen, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl;
or a pharmaceutically acceptable salt thereof;
with the proviso that R₁ and R₂ are not simultaneously hydrogen;

41. The method of claim 40, wherein R₃ is phenyl or a phenyl group substituted with 1 to 5 substituents selected from the group consisting of halo, hydroxy, aryl, C₁-C₆ alkyl substituted aryl, nitro, polycyclic aryl alkyl containing 2 to 4 aromatic rings wherein the alkyl is a C₁-C₆, C₃-C₈ cycloalkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, aryloxy, acyloxy, acyloxy C₁-C₆ alkyl, amino, monoalkylamino wherein the alkyl is C₁-C₆, dialkylamino wherein the alkyl is C₁-C₆, acylamino, ureido, thioureido, carboxy, carboxy C₁-C₆ alkyl, azido, cyano, cyano C₁-C₆ alkyl, formyl, acyl, dialkoxy alkyl wherein the alkoxy and alkyl are independently C₁-C₆, aminoalkyl wherein the alkyl is C₁-C₆, and SOₙR′ wherein n=0, 1, 2 or 3, R′ is H, a C₁-C₆ alkyl or aryl; or a pharmaceutically acceptable salt thereof.

42.-48. (canceled)
